(19) United States
(12) Patent Application Publication
(10) Pub. No.: US 2013/0204017 A1
(43) Pub. Date: Aug. 8, 2013

(54) GLYCERYL ASCORBIC ACID ACYLATED DERIVATIVE OR ITS SALT, PRODUCTION METHOD THEREOF, AND COSMETICS

(71) Applicants: Masato YOSHIOKA, Higashiosaka-shi (JP); Norihisa TAIRA, Higashiosaka-shi (JP)

(72) Inventors: Masato YOSHIOKA, Higashiosaka-shi (JP); Norihisa TAIRA, Higashiosaka-shi (JP)

(73) Assignee: SEIWA KASEI COMPANY, LIMITED, Higashiosaka-shi (JP)

(21) Appl. No.: 13/364,892

(22) Filed: Feb. 2, 2012

(51) Int. Cl. C07D 307/62

(52) U.S. Cl. 549/477

(57) ABSTRACT

A glyceryl ascorbic acid acylated derivative or its salt, which has an ascorbic acid structure where 2- and/or 3-positions of the structure are substituted with glyceryl groups and some of the hydroxyl groups in the structure and/or in the glyceryl group are acylated, a production method of the glyceryl ascorbic acid acylated derivative and a cosmetic containing the glyceryl ascorbic acid acylated derivative or its salt are provided.

### TECHNICAL FIELD

The present invention relates to a glyceryl ascorbic acid acylated derivative or its salt which is suitably used as a raw material of a cosmetic and the like. Also, the present invention relates to a production method of above-described glyceryl ascorbic acid acylated derivative or its salt. Further, the present invention relates to cosmetics comprising the above-described glyceryl ascorbic acid acylated derivative or its salt.

### BACKGROUND ART

Ascorbic acid is a safe and useful substance, is known as a compound having an antioxidant effect, a collagen production promoting effect, an excellent whitening effect and the like. However, ascorbic acid is particularly unstable to light, heat and oxidation, thus preventing utilization thereof in cosmetics, food products, pharmaceuticals and the like. As materials having improved stability over time than ascorbic acid, various ascorbic acid derivatives or salts thereof are proposed. Their compounding in a skin external agent for whitening, their utilization as a moisturizer, and their compounding in cosmetics for promoting collagen production and the like are also proposed. (Patent document 1, Patent document 2).

Among ascorbic acid derivatives described in patent document 1, however, particularly glyceryl ascorbic acid excellent in a moisturizing effect is water-soluble, and, thus, has a problem that permeability thereof is low and it does not easily get to the intended tissue, when it is applied to a hydrophobic region such as skin, mucous membrane and the like.

For solving this problem, patent document 1 discloses a compound having a long chain alkyl group ether-linked to a glycerin structure, and the like. In this compound, however, the alkyl group is ether-linked simply to a glyceryl group, thus, glyceryl ascorbic acid is not liberated easily in a living organism and the moisturizing effect of glyceryl ascorbic acid cannot be expected inside the skin. In addition, its production cost is high.

Patent document 3 and patent document 4 disclose acylated derivatives of glucosyl-L-ascorbic acid, and describe that the acylated derivatives are excellent in a radical scavenging ability and an anti-scorbutic property. However, a moisturizing effect is not disclosed at all, and it is hard to expect a moisturizing effect contributing significantly to flexibility of skin, improvement of skin roughness and the like.

Then, there is a desire for development of a glyceryl ascorbic acid derivative which has the moisturizing effect of glyceryl ascorbic acid, has excellent permeability into skin, additionally, is excellent in a whitening effect, a collagen production promoting effect and the like, and can be produced at low cost.

### PRIOR ART DOCUMENT

### Patent document

(Patent document 1) WO 2009/025328
(Patent document 2) JP-A No. 2005-60239
(Patent document 3) JP-A No. 11-286497
(Patent document 4) Japanese Patent No. 4307784

### SUMMARY OF THE INVENTION

### Problem to be Solved by the Invention

An object of the present invention is to provide a novel glyceryl ascorbic acid derivative or its salt having an excellent function which glyceryl ascorbic acid has, also excellent in skin permeability, and further excellent in a moisturizing effect, a feeling of use, a collagen production promoting effect, a whitening effect and the like. Another object of the present invention is to provide a production method which is capable of producing the novel glyceryl ascorbic acid derivative or its salt easily at low cost. Further, another object of the present invention is to provide cosmetics comprising this novel glyceryl ascorbic acid derivative or its salt and having an excellent function.

### Means for Solving the Problem

The present inventors have intensively investigated in view of the above-described problems and resultantly found that a glyceryl ascorbic acid acylated derivative shows excellent solubility also in an oil-soluble substance and has a high moisturizing property in a living body. Further, it has been found that this glyceryl ascorbic acid acylated derivative can be obtained by reacting glyceryl ascorbic acid with an acylation agent. These glyceryl ascorbic acid acylated derivatives can be used for preparation of cosmetics and can be used as a food additive, a feedstuff and the like. The present invention has been completed based on these findings.

In glyceryl ascorbic acid referred to in the present invention, a glyceryl group is linked to any oxygen atom or several oxygen atoms of hydroxyl groups of ascorbic acid, and the glyceryl group is represented by HO—CH₂—CH(OH)—CH₂—. Specific examples of glyceryl ascorbic acid include 2-O-glyceryl ascorbic acid, 3-O-glyceryl ascorbic acid and 2,3-di-O-glyceryl ascorbic acid. These glyceryl ascorbic acids can be produced by a method described in Patent document 1, and the like. The above-described glyceryl ascorbic acid acylated derivative is a derivative in which an acyl group is ester-linked to a hydroxyl group of glyceryl ascorbic acid, thereby enhancing hydrophobicity.

That is, the present invention provides a glyceryl ascorbic acid acylated derivative represented by the following general formula (I) or its salt. (claim 1)

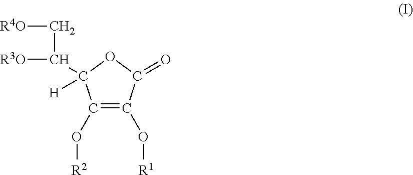

[wherein, R¹ and R² represent a hydrogen atom, R⁵—O—CH₂—CH(OH)—CH₂— or an acyl group represented by R⁶—CO—, R³ and R⁴ represent a group represented by R⁵—, here, R⁵ represents a hydrogen atom or an acyl group represented by R⁶—CO—, and R⁶ represents a hydrogen atom, an alkyl group having 1 to 22 carbon atoms or an alkenyl group having 2 to 22 carbon atoms, provided that, at least one of R¹— and R² is a group represented by R⁵—O—CH₂—CH(OH)—CH₂—, and when both R³ and R⁴ are a hydrogen atom, at least one of R¹— and R² is a group represented by R⁶—CO—O—CH₂—CH(OH)—CH₂—.]

The invention of claim 2 is a glyceryl ascorbic acid acylated derivative or its salt according to claim 1, in which R¹ and R² represent a hydrogen atom or R⁵—O—CH₂—CH(OH)—CH₂—, R³ represents a hydrogen atom and R⁴ represents a hydrogen atom or an acyl group represented by R⁶—CO—. That is, in the glyceryl ascorbic acid acylated derivative of claim 2, a hydrogen atom is linked to oxygen at 5-position of an ascorbic acid structure. This glyceryl ascorbic acid acylated derivative gives good sensory textures to skin. Specially, it provides good moist feel without uncomfortable sticky and frictional feel.

The invention of claim 3 is a glyceryl ascorbic acid acylated derivative or its salt according to claim 2 in which only one of R¹, R² and R⁴ represents R⁶—CO—. That is, in the glyceryl ascorbic acid acylated derivative of claim 3, only one hydroxyl group among hydroxyl groups of glyceryl ascorbic acid is linked to an acyl group represented by R⁶—CO—. This glyceryl ascorbic acid acylated derivative is excellent also in water-solubility and dispersibility, and can also be compounded at high concentration in cosmetics such as a skin lotion and the like.

The invention of claim 4 is a glyceryl ascorbic acid acylated derivative or its salt according to claim 1, in which R¹ and R² represent a hydrogen atom or R⁵—O—CH₂—CH(OH)—CH₂—, R³ and R⁵ represent a hydrogen atom, and R⁴ represents R⁶—CO—. That is, in the glyceryl ascorbic acid acylated derivative or its salt of claim 4, only a hydrogen of hydroxyl group at 6-position of an ascorbic acid structure of glyceryl ascorbic acid is substituted by an acyl group. This glyceryl ascorbic acid acylated derivative improves the stability of an emulsion such as a cream and a milky lotion.

The invention of claim 5 is a glyceryl ascorbic acid acylated derivative or its salt according to claim 1, in which R¹ represents R⁵—O—CH₂—CH(OH)—CH₂—, R², R³ and R⁵ represent a hydrogen atom, and R⁴ represents R⁶—CO—. That is, in the glyceryl ascorbic acid acylated derivative or its salt of claim 5, a hydrogen of hydroxyl group at 6-position of an ascorbic acid structure of 2-O-glyceryl ascorbic acid is substituted by an acyl group. Due to the linking of glycerin to 2-position of an ascorbic acid structure, stability over time, emulsion stability, no color change with time course and the like are excellent when compounded into cosmetics. Further, a hydroxyl group at 3-position can be converted into a neutral salt by a known method, and water-solubility and dispersibility can be improved.

The invention of claim 6 is a glyceryl ascorbic acid acylated derivative or its salt according to claim 5, in which R⁶ represents an alkyl group having 7 to 22 carbon atoms or an alkenyl group having 7 to 22 carbon atoms. This 2-O-glyceryl ascorbic acid acylated derivative or its salt exhibits a particularly excellent melanin production suppressing effect.

The invention of claim 7 is a glyceryl ascorbic acid acylated derivative or its salt according to claim 6, in which R⁶ is an alkyl group having 11, 13, 15 or 17 carbon atoms. This 2-O-glyceryl ascorbic acid acylated derivative or its salt is a further preferable embodiment of the invention of claim 6.

The invention of claim 8 is a glyceryl ascorbic acid acylated derivative or its salt according to claim 1, in which R¹ represents R⁵—O—CH₂—CH(OH)—CH₂—, R² and R³ represent a hydrogen atom, and R⁴ and R⁵ represents R⁶—CO—. That is, in the glyceryl ascorbic acid acylated derivative or its salt of claim 8, an acyl group represented by R⁶—CO— is ester-linked to hydroxyl groups at 6-position of an ascorbic acid structure and 3′-position of a glyceryl group of 2-O-glycerylascorbic acid. Each hydrogen atom of hydroxyl groups at two positions of 2-O-glyceryl ascorbic acid is substituted by an acyl group, two hydrophobic functional groups are carried on both sides of the molecular structure, and a hydrophilic functional group is contained in its center. Therefore, when compounded into an emulsifying cosmetic, the stability of the cosmetic is enhanced.

The invention of claim 9 is a glyceryl ascorbic acid acylated derivative or its salt according to claim 8, in which R⁶ represents an alkyl group having 7 to 22 carbon atoms or an alkenyl group having 7 to 22 carbon atoms. This glyceryl ascorbic acid acylated derivative or its salt is a more preferable embodiment of the acylated body of 2-O-glyceryl ascorbic acid according to claim 8, and exhibits a particularly excellent collagen production promoting effect.

The invention of claim 10 is a glyceryl ascorbic acid acylated derivative or its salt according to claim 1, in which R¹ represents R⁵—O—CH₂—CH(OH)—CH₂—, R³ represents a hydrogen atom, and R², R⁴ and R⁵ represent R⁶—CO—. That is, an acylated body of 2-O-glyceryl ascorbic acid in which an acyl group represented by R⁶—CO— is ester-linked to hydroxyl groups at 3-position and 6-position of an ascorbic acid structure and at 3′-position of a glyceryl group of 2-O-glyceryl ascorbic acid, or its salt. In this acylated body, each hydrogen atom of hydroxyl groups at three positions of 2-O-glyceryl ascorbic acid is substituted by an acyl group, and the acylated body is excellent particularly in solubility in an oil component. Therefore, it can be compounded easily in an oil phase in producing an emulsion.

Further, the present invention provides a production method of a glyceryl ascorbic acid acylated derivative or its salt comprising reacting a glyceryl ascorbic acid with an acylating agent (claim 11). By this production method, the glyceryl ascorbic acid acylated derivative of the present invention can be produced easily.

The invention of claim 12 is a production method of a glyceryl ascorbic acid acylated derivative or its salt according to claim 11, in which the acylating agent is an acid chloride or an acid anhydride and the reaction is carried out under basic conditions. By this method of claim 12, the glyceryl ascorbic acid acylated derivative of the present invention can be produced more easily.

The invention of claim 13 is a production method of a glyceryl ascorbic acid acylated derivative or its salt according to claim 11, in which the acylating agent is a carboxylic acid and the reaction is carried out under acidic conditions. By this method of claim 13, the glyceryl ascorbic acid acylated derivative of the present invention can be produced at a low cost.

Still further, the present invention provides a cosmetic comprising the glyceryl ascorbic acid acylated derivative or its salt according to any one of claims 1 to 10 (claim 14). The glyceryl ascorbic acid acylated derivative or its salt of the present invention has excellent functions such as a moisturizing effect, a good sensory texture upon use, a rough skin preventing effect, an anti-aging effect, a collagen production promoting effect, a whitening effect and the like, thus, the cosmetic of the present invention also has these excellent functions.

### Effect of the Invention

The above-described glyceryl ascorbic acid acylated derivative represented by the general formula (I) or its salt of the present invention has excellent functions of glyceryl ascorbic acid such as a whitening effect, a collagen production promoting effect and the like. Additionally, it has a moisturizing effect and shows high permeability and stability, and it shows low color change, low odor generation, degradation and small decrease in activity and the like even in storage for a long period of time. Therefore, by compounding this compound in a cosmetic such as a skin external agent, a hair cosmetic and the like, a cosmetic excellent in a whitening effect, a moisturizing effect and the like and having high stability even in storage for a long period of time can be obtained.

### MODES FOR CARRYING OUT THE INVENTION

The glyceryl group referred to in the present invention is a group represented by HO—CH₂—CH(OH)—CH₂—. In glyceryl ascorbic acid, the glyceryl group is linked to any oxygen atom or several oxygen atoms of hydroxyl groups of ascorbic acid. The glyceryl ascorbic acid acylated derivative of the present invention includes all glyceryl ascorbic acid acylated derivatives endowed with hydrophobicity by acylation. Glyceryl ascorbic acid which is particularly preferable for the acylated derivative is 2-O-glyceryl ascorbic acid in which a glyceryl group (HO—CH₂—CH(OH)—CH₂—) is linked to the oxygen of a hydroxyl group at 2-position of ascorbic acid.

The acylation referred to in the present invention means to introduce an acyl group (R⁶—CO—) into glyceryl ascorbic acid. Here, R⁶ is a linear or branched saturated or unsaturated alkyl or alkenyl group. Generally as R⁶, an alkyl group having 3 to 17 carbon atoms is preferable, and an alkyl group having 7 to 17 carbon atoms is more preferable. Therefore, the glyceryl ascorbic acid acylated derivative of the present invention includes overall compounds in which an acyl group is linked to one or several hydroxyl groups in glyceryl ascorbic acid. Among them, a glyceryl ascorbic acid acylated derivative in which an acyl group is linked to one hydroxyl group in an ascorbic acid structure of glyceryl ascorbic acid is desirable.

Specific examples of the glyceryl ascorbic acid acylated derivative of the present invention include compounds described below, but the scope of the present invention is not limited to the following compounds.

Monoacylglyceryl ascorbic acids;
2-O-glyceryl-6-O-acyl ascorbic acid,
2-O-glyceryl-5-O-acyl ascorbic acid,
2-O-glyceryl-3-O-acyl ascorbic acid,
2-O-(3′-O-acylglyceryl) ascorbic acid,
3-O-glyceryl-6-O-acyl ascorbic acid,
3-O-glyceryl-5-O-acyl ascorbic acid,
3-O-glyceryl-2-O-acyl ascorbic acid,
3-O-(3′-O-acylglyceryl) ascorbic acid, and the like;
   Diacylglyceryl ascorbic acids;
2-O-acylglyceryl-6-O-acyl ascorbic acid, and the like;
   Triacylglyceryl ascorbic acids
2-O-acylglyceryl-3-O-acyl-6-O-acyl ascorbic acid, and the like;

The acyl group in the examples mentioned above means a formyl group, an acetyl group, a propionyl group, a butanoyl group, a pentanoyl group, a hexanoyl group, a heptanoyl group, an octanoyl group, a nonanoyl group, a decanoyl group, an undecanoyl group, a dodecanoyl group, a tetradecanoyl group, a hexadecanoyl group, an octadecanoyl group, an eicosanoyl group, a hexadecenoyl group, an octadecenoyl group, an ° leyl group, an octadecatrienoyl group, an isooctanoyl group, an isopalmitoyl group, an isostearoyl group, a 2-propylpentanoyl group, a 2-butylhexanoyl group, a 2-pentylheptanoyl group or the like. Acyl groups in the di- and tri-acylated derivatives may be the same or different.

According to the production method of the present invention, the above-described glyceryl ascorbic acid acylated derivative represented by the general formula (I) or its salt can be produced only by simply reacting glyceryl ascorbic acid with an acylation agent. Therefore, the glyceryl ascorbic acid acylated derivative or its salt of the present invention can be produced easily at low cost.

Examples of the acylation agent used in the production method of the present invention include; carboxylic acids such as acetic acid, propionic acid, butyric acid, isobutyric acid, valeric acid, isovaleric acid, trimethylacetic acid, caproic acid, enanthic acid, caprylic acid, pelargonic acid, capric acid, lauric acid, myristic acid, pentadecylic acid, palmitic acid, margaric acid, stearic acid, oleic acid, vaccenic acid, linoleic acid, linolenic acid, ricinoleic acid, arachidonic acid, petroselic acid, eleostearic acid, licanic acid, parinaric acid, tariric acid, gadoleic acid, behenic acid, lignoceric acid, nervonic acid, melissic acid and the like; halide of the above exemplified carboxylic acids; anhydrides of the above exemplified carboxylic acids; esters of the above exemplified carboxylic acids; amides of the above exemplified carboxylic acids; and the like.

The acylation reaction of the present invention can be carried out without solvent or in various solvents. Examples of the solvents include

protic solvents such as water and lower alcohols including methanol, ethanol, isopropanol and the like;
aprotic solvents such as dimethyl sulfoxide (DMSO), N,N-dimethylformamide (DMF), pyridine, dioxane, tetrahydrofuran (THF) and the like; and
mixed solvents thereof. Among the above, aprotic solvents are preferable.

Though the reaction temperature is not particularly restricted, it is preferably 0 to 100° C. for accelerating the reaction of glyceryl ascorbic acid with an acylation agent. It is preferably 20 to 60° C. for obtaining regioselectivity in the reaction. Though pH of the reaction solvent is not particularly restricted, a basic condition is preferable in the case of effecting the reaction using an acid halide or an acid anhydride, and in this case pH of 10 or more is more preferable. In contrast, an acidic condition is preferable in the case of effecting the reaction using a carboxylic acid, and in this case pH of 3 or less is more preferable.

Since ascorbic acids are easily oxidized, it is preferable to substitute the atmosphere in the reaction system with an inert gas such as argon, nitrogen or helium, although the glyceryl ascorbic acid acylated derivative of the present invention can be obtained by a reaction carried out without substituting the reaction system with an inert gas. By carrying out the reaction under an inert gas atmosphere, color change, odor change and the like can be lowered.

In addition to the above-mentioned reactive substance, substances exemplified following can be added to the reaction system:

basic substances such as sodium hydroxide, sodium hydrogen carbonate, ammonia, triethylamine, triethanolamine, and the like;
acidic substances such as hydrochloric acid, sulfuric acid, and the like;
metals such as aluminum chloride, and the like;
a phase transfer catalyst such as tetrabutyl ammonium bromide;
enzymes such as Lipase, and the like.

The above substances may be dissolved or dispersed in a small amount of solvent such as DMF before addition, for sufficient mixing thereof. Though the method of mixing raw materials such as a glyceryl ascorbic acid and an acylation agent is not particularly restricted, it is also possible to drop the acylation agent into the reaction system.

Though the use amount of the acylation agent with respect to the glyceryl ascorbic acid is not particularly restricted, it is preferably 0.5 to 5 mol with respect to 1 mol of glyceryl ascorbic acid. When the use amount of the acylation agent with respect to the glyceryl ascorbic acid is smaller, the position-selectivity of the addition reaction, that is, selective addition to 6-position is more remarkable. In the case of addition to only 6-position, it is preferably in the range of about 0.5 to 1.5 mol.

A compound in which acyl groups are added to both the hydroxyl group at 6-position of ascorbic acid structure and the hydroxyl group at 3′-position of glycerin structure can be obtained, for example, by a method in which

the acylation reaction is carried out under the above-mentioned conditions (that is, the use amount of the acylation agent is in the range of about 0.5 to 1.5 mol) to obtain 6-acyl-glycerylascorbic acid; then,
purification is performed to remove unreacted glyceryl ascorbic acid, followed by reacting the 6-acyl-glycerylascorbic acid with about 0.5 to 1.5 mol of acylation agent. By changing the acylation agent to be used in the reaction before purification and the reaction after purification, a compound in which different acyl groups are added to both the hydroxyl group at 6-position of ascorbic acid structure and the hydroxyl group at 3′-position of glycerin structure can be obtained.

The glyceryl ascorbic acid acylated derivative or its salt produced as described above can be purified by means such as column chromatography using silica gel or a resin such as an ion exchange resin, treatment with activated carbon, extraction, distillation, crystallization and the like.

In the glyceryl ascorbic acid acylated derivative of the above-described formula (I) in which R¹ or R² is hydrogen, the hydrogen can be substituted by a positive ion such as a metal ion, ammonium ion and the like to form a salt of glyceryl ascorbic acid acylated derivative, and this salt is also included in the scope of the present invention. This salt includes inorganic salts and organic salts. Inorganic salts include salts of an alkali metal such as sodium and potassium, salts of an alkaline earth metal such as calcium, magnesium salt, ammonium salt and the like. Organic salts include a diethanolamine salt, triethanolamine salt, basic amino acid salt and the like. Formation of the salt can be carried out by the same method as for known salt formation method such as neutralization of an aqueous solution of a glyceryl ascorbic acid acylated derivative in which R¹ or R² is H with a basic substance.

The glyceryl ascorbic acid acylated derivative or its salt of the present invention is suitably used as a component of various cosmetics such as skin or hair cosmetics.

The glyceryl ascorbic acid acylated derivative or its salt of the present invention has excellent effects such as a whitening effect, a collagen production promoting effect and the like which ascorbic acid basically has, and additionally, has a moisturizing effect. It shows excellent stability and low color change, low odor generation, small decrease in activity, and the like even in storage for a long period of time. Then, cosmetics containing this glyceryl ascorbic acid acylated derivative or its salt as a component have an excellent whitening effect, a collagen production promoting effect, a moisturizing effect and the like, and additionally, are excellent also in stability over time.

Further, there can be obtained cosmetics which can be expected to have excellent effects such as an anti-aging effect, a skin photo-aging preventing effect, a Maillard reaction inhibiting effect, a wrinkle formation inhibiting effect, an antioxidant effect, a blood circulation promoting effect and the like, and are stable even in storage for a long period of time. The glyceryl ascorbic acid acylated derivative or its salt of the present invention can be utilized also as a food additive, a feedstuff, a pharmaceutical product or the like.

The compounding amount of a glyceryl ascorbic acid acylated derivative or its salt of the present invention in various cosmetics is not particularly restricted, since it varies depending on the application of a cosmetic. In most cases, it is preferably in the range of 0.01 to 20% by mass. In many cases, when it is less than 0.01% by mass, the effects of the glyceryl ascorbic acid acylated derivative or its salt of the present invention, such as whitening effect, cannot be manifested sufficiently. In contrast, when it is over 20% by mass, an effect corresponding to the compounding amount cannot be obtained in most cases. Further, the cosmetics may lose stability in appearance.

In the cosmetic of the present invention, components usually used for cosmetics, for example, oily materials, surfactants, moisturizing agents, polymers, antioxidants, whitening agents, medicines, ultraviolet absorbers, metal ion sequestrants, and the like can be appropriately compounded, in addition to the essential components. Though the glyceryl ascorbic acid acylated derivative or its salt of the present invention acts also as a moisturizing agent, other moisturizing agents can be appropriately compounded into the cosmetic of the present invention.

Examples of the oily materials include

oils and fats such as olive oil, camellia oil, macadamia nut oil, tea oil, castor oil and tri(caprone/capryl)glyceryl;
waxes such as jojoba oil, carnauba wax, candelilla wax, lanolin and bees wax;
hydrocarbons such as liquid paraffin, paraffin, vaseline, seresin, microcrystalline wax and squalane,
fatty acids such as lauric acid, myristic acid, palmitic acid, stearic acid, behenic acid and isostearic acid;
higher alcohols such as cetyl alcohol, stearyl alcohol and isostearyl alcohol;
esters such as isopropyl myristate, 2-octyldodecyl myristate, cetyl 2-ethylhexanoate, diisostearyl malate and tri-2-ethylhexanoin; and
silicones such as methyl polysiloxane, methyphenyl polysiloxane and decamethyl cyclopenta siloxane.

Examples of the surfactants include

anionic surfactants such as higher fatty acid soaps, polyoxyethylene alkyl ether sulfate, acyl-N-methyl taurate, N-acyl amino acid salts and alkyl phosphates;
cationic surfactants such as alkyl trimethyl ammonium chloride and dialkyl dimethyl ammonium chloride;
ampholytic surfactants such as alkyl dimethyl aminoacetic acid betaine, alkyl amide aminoacetic acid betaine and 2-alkyl-N-carboxy-N-hydroxy imidazolynium betaine; and
nonionic surfactants such as polyoxyethylene alkyl ether, polyethylene glycol fatty acid ester, poly-hydric alcohol fatty acid ester and polyether-modified silicone.

Examples of the other moisturizing agents include glycerin, propylene glycol, maltitol, sorbitol, 1,3-butylene glycol, sodium lactate, polyethylene glycol, sodium pyrrolidone carboxylate and sodium hyaluronate.

Examples of the polymer compounds include carboxy vinyl polymer, carboxy methylcellulose sodium, xanthan gum, polyvinyl alcohol and dimethylpolysiloxane polymer.

Examples of the antioxidants include vitamin E, tannin and BHT (butylhydroxytoluene).

Examples of the other whitening agents include ellagic acid, chamomile extract, liquorice extract, rucinol, rosemary extract, arbutin, tranexamic acid, potassium 4-methoxysalicylate, ascorbic acid; ascorbic acid derivatives such as glucosyl ascorbic acid and magnesium ascorbyl phosphate; and the like.

The other agents include rough skin preventing agents and anti-inflammatory agents. Examples of the rough skin preventing agent and anti-inflammatory agent include dipotassium glycyrrhizinate, steary glycirrhetinate, methyl salicylate, pyridoxine hydrochloride, allantoin, marine salt, mulberry root extract, aloe extract, gardenia florida extract, chamomile extract, liquorice extract, soapberry peel extract, apricot kernel extract, scutellaria root extract, sweet tea extract, loquat extract, ginkgo biloba extract, hypericum extract, yarrow extract, safflower extract, bitter orange peel extract, sage leaf extract, birch extract, citrus unshiu peel extract, peach kernel extract, mugwort extract, althea extract, arnica extract, ginseng extract, paeony root extract, cnidium officinale root extract, gentian extract, cordyceps sinensis extract, phellodendron bark extract, artemisia capillaris flower extract, geranium thunbergi extract, peach leaf extract, sasa albo-marginata extract, job's tears extract, horse chestnut extract, crataegus cuneata fruit extract, coptis japonica root extract, mushroom extract, calendula officinalis extract, peppermint extract, symphytum officinale extract, butcher's broom extract, malva sylvestris flower extract, rodgersia podophylla extract, rosa roxburghii fruit extract and the like. Additionally, an agent for hair growth, an agent for acne, an agent for dandruff and itching, an underarm odor-preventing agent and the like are also listed as the other agents.

Examples of the hydrolyzed protein include protein hydrolysates such as milk protein, silk protein, wheat protein, rice protein, pea protein, collagen, keratin, soybean, sesame, conchiolin, marine collagen and the like, and derivatives thereof, and the like.

Examples of the amino acid or its derivative include amino acids such as glycine, valine, leucine, isoleucine, serine, threonine, phenylalanine, arginine, lysine, asparagine, aspartic acid, glutamine, glutaminic acid, cystine, cysteine, methionine, tryptophan, proline, histidine and the like, and derivatives thereof.

Examples of the pH regulator include lactic acid, citric acid, glycolic acid, succinic acid, tartaric acid, malic acid, potassium carbonate, sodium hydrogen carbonate, ammonium hydrogen carbonate and the like.

Examples of the preservative include alkyl p-oxybenzoates, benzoic acid, sodium benzoate, sorbic acid, potassium sorbate, phenoxyethanol and the like.

Examples of the thickening agent include gum Arabic, tragacanth gum, carob gum, guar gum, pectin, agar, quince seed, starch, algae colloid, xanthan gum, dextran, succinoglucan, collagen, gelatin, casein, albumin, carboxymethyl starch, methylcellulose, ethylcellulose, methylhydroxypropylcellulose, carboxymethylcellulose, hydroxymethylcellulose, hydroxypropylcellulose, nitrocellulose, sodium cellulose sulfate, sodium carboxymethylcellulose, sodium alginate, polyvinyl methyl ether, carboxy vinyl polymer, sodium polyacrylate, polyethylene acrylate, polyacrylamide, cation polymer and the like.

Examples of the coloring matter include tar dye, natural colorants, inorganic pigments, polymer powders and the like. Examples of the perfume include natural perfumes, synthetic perfumes, blended perfumes and the like.

The form of the cosmetic of the present invention is arbitrary. Any of a solution system, solubilization system, emulsion system, gel system, powder dispersion system, water-oil two-layer system and the like are possible. According to the intended cosmetic product, a glyceryl ascorbic acid acylated derivative or its salt of the above-described general formula (I) and the above-described optional compounding components can be compounded.

### EXAMPLES

Next, specific embodiments for carrying out the present invention will be explained concretely by examples. The scope of the present invention is not limited to the examples. First, examples for producing glyceryl ascorbic acids used in Examples and Comparative examples are described as Synthesis Examples.

### Synthesis Example 1

### Synthesis of 2-O-glyceryl ascorbic acid

Under an argon atmosphere, to water were added L-ascorbic acid (10.0 g) and sodium hydrogen carbonate (9.54 g), the mixture was stirred at room temperature for 30 minutes, then glycidol (8.41 g) was added. The mixture was heated up to 60° C. and stirred for 5 hours. Methanol was added and the mixture was filtrated, the filtrate was concentrated under reduced pressure, and 19.0 g of the resultant residue was subjected to silica gel column chromatography. Elution was performed with chloroform/methanol/water=6/4/1, and the eluate was concentrated under reduced pressure, to obtain 2-O-glyceryl ascorbic acid (1.21 g).

The resultant product was subjected to ¹H-NMR and ¹³C-NMR measurement, and based on the measured results, it was confirmed that this product was 2-O-glyceryl ascorbic acid represented by the following structural formula. Also in examples shown below, the resultant product was subjected to

¹H-NMR and/or ¹³C-NMR measurement, and the measured results were shown for the examples.

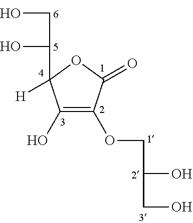 [SMILES: O=C1OC(C(O)CO)C(O)=C1OCC(O)CO]

In this formula, carbon atoms, and hydrogen atoms linked to carbon atoms are abbreviated. For example, in this formula, 1 to 4-positions are carbon atoms, 6, 1′ and 3′-positions are CH₂ groups, and 5 and 2′-positions are CH groups. The same shall apply also in the following formulae.

The analyzed results by NMR were as described below. ¹H-NMR (400 MHz, CD₃OD): δ ppm 3.61 (2H, m), 3.67 (2H, m), 3.90 (1H, m), 3.92 (1H,dt-like), 3.92 (1H, m), 4.07/4.09 (1H, dd), 4.86 (1H, d)

¹³C-NMR (100 MHz, CD₃OD): δ ppm 63.3, 63.7, 70.4, 72.0, 74.6, 76.8, 122.2, 161.6, 172.9

### Synthesis Example 2

### Synthesis of 3-O-glyceryl ascorbic acid

Under an argon atmosphere, to water were added L-ascorbic acid (300 g) and sodium hydrogen carbonate (42.9 g), the mixture was stirred at room temperature for 30 minutes, then, glycidol (126 g) was added. Thereafter, the mixture was heated up to 50° C. and stirred for 5 hours. Methanol was added and the mixture was filtrated, the filtrate was concentrated under reduced pressure, and 457 g of the resultant residue was subjected to silica gel column chromatography. Elution was performed with chloroform/methanol/water(=65/35/5), and the eluate was concentrated under reduced pressure, to obtain 3-O-glyceryl ascorbic acid (296 g) represented by the following formula.

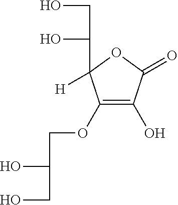 [SMILES: O=C1OC(C(O)CO)C(OCC(O)CO)=C1O]

The analyzed results by NMR were as described below.

¹H-NMR (600 MHz, CD₃OD): δ ppm 3.59 (2H, m), 3.66 (2H, m), 3.89 (1H, m), 3.92 (1H, m), 4.45/4.49 (1H, dd), 4.59/4.62 (1H, dd), 4.82 (1H, d)

¹³C-NMR (150 MHz, CD₃OD): δ ppm 63.4, 63.7, 70.56, 70.61, 71.79, 71.89, 73.4, 73.6, 76.9, 121.17, 121.24, 151.84, 151.88, 173.04, 173.07

### Example 1

### Synthesis of 2-O-glyceryl-6-O-butanoyl ascorbic acid

Under an argon atmosphere, to 2-O-glyceryl ascorbic acid (50 mg) were added 5 mL of pyridine and n-butanoic anhydride (57 mg), and the mixture was stirred for 3 hours at 60° C. Thereafter, ethyl acetate was added and the mixture was extracted with water. The extracted liquid was concentrated under reduced pressure, and 98 mg of the resultant residue was subjected to silica gel column chromatography. Purification was performed by eluting with mixed liquid of chloroform/methanol/water(=7/3/0.3), and the eluate was concentrated under reduced pressure, to obtain a reaction product (43 mg). Identification by ¹H-NMR and ¹³C-NMR confirmed that the product was 2-O-glyceryl-6-O-butanoyl ascorbic acid represented by following chemical formula.

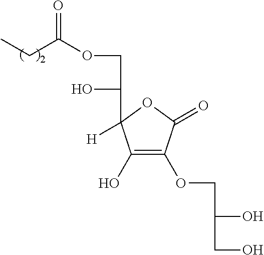

The analyzed results by NMR were as described below.

¹H-NMR (400 MHz, CD₃OD): δ ppm 0.95 (3H, t), 1.60 (2H, m), 2.34 (2H, m), 3.60 (2H, t), 3.90 (2H, m), 4.13 (3H, m), 4.79 (1H, d)

¹³C-NMR (100 MHz, CD₃OD): δ ppm 13.9, 19.3, 36.7, 63.7, 65.6, 67.9, 72.0, 74.65, 74.70, 77.19, 122.29, 122.32, 161.50, 161.54, 172.6, 174.9

### Example 2

### Synthesis of 2-O-glyceryl-6-O-octanoyl ascorbic acid

Under an argon atmosphere, to 2-O-glyceryl ascorbic acid (50 mg) were added 5 mL of pyridine and n-octanoic anhydride (97 mg), and the mixture was stirred for 3 hours at 60° C. Thereafter, ethyl acetate was added and the mixture was extracted with water. The extracted liquid was concentrated under reduced pressure, and 134 mg of the resultant residue was subjected to silica gel column chromatography. Purification was performed by eluting with mixed liquid of chloroform/methanol/water(=7/3/0.3), and the eluate was concentrated under reduced pressure, to obtain a reaction product (67 mg). Identification by ¹H-NMR and ¹³C-NMR confirmed that the product was 2-O-glyceryl-6-O-octanoyl ascorbic acid represented by following chemical formula.

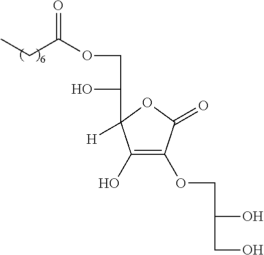

The analyzed results by NMR were as described below.

¹H-NMR (400 MHz, CD₃OD): δ ppm 0.89 (3H, t), 1.31 (8H, m), 1.60 (2H, m), 2.36 (2H, m), 3.60 (2H, t), 3.87 (1H, m), 3.94 (1H, m), 4.09 (2H, m), 4.17 (1H, m), 4.27 (1H, m), 4.79 (1H, d)

¹³C-NMR (100 MHz, CD₃OD): δ ppm 14.4, 23.6, 25.9, 30.0, 30.1, 32.8, 34.8, 63.8, 65.6, 67.9, 72.1, 74.70, 74.74, 77.2, 122.4, 161.3, 172.6 175.4

### Example 3

### Synthesis of 2-O-glyceryl-6-O-hexadecanoyl ascorbic acid

Under an argon atmosphere, to 2-O-glyceryl ascorbic acid (50 mg) were added 5 mL of pyridine and n-hexadecanoic anhydride (176 mg), and the mixture was stirred for 3 hours at 60° C. Thereafter, water was added and the mixture was extracted with ethyl acetate. The extracted liquid was concentrated under reduced pressure, and 134 mg of the resultant residue was subjected to silica gel column chromatography. Purification was performed by eluting with mixed liquid of chloroform/methanol/water(=7/3/0.3), and the eluate was concentrated under reduced pressure, to obtain a reaction product (65 mg). Identification by ¹H-NMR and ¹³C-NMR confirmed that the product was 2-O-glyceryl-6-β-hexadecanoyl ascorbic acid represented by following chemical formula.

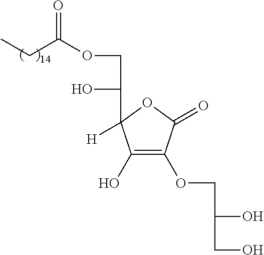

The analyzed results by NMR were as described below.

¹H-NMR (400 MHz, CD₃OD): δ ppm 0.95 (3H, t), 1.31 (24H, m), 1.6 0 (2H, m), 2.34 (2H, m), 3.60 (2H, t), 3.90 (2H, m), 4.13 (3H, m), 4.79 (1H, d)

¹³C-NMR (100 MHz, CD₃OD): δ ppm 14.4, 23.7, 26.0, 30.2, 30. 4, 30.5, 30.7, 33.0, 35.0, 63.7, 65.6, 67.8, 71.9, 74.7, 74.8, 7 7.3, 122.3, 161.6, 172.9, 175.1

### Example 4

### Synthesis of 2-O-(3′-O-octanoylglyceryl)-6-O-octanoyl ascorbic acid

Under an argon atmosphere, to 2-O-glyceryl ascorbic acid (50 mg) were added 5 mL of pyridine and n-octanoic anhydride (194 mg), and the mixture was stirred for 3 hours at 60° C. Thereafter, water was added and the mixture was extracted with ethyl acetate. The extracted liquid was concentrated under reduced pressure, and 232 mg of the resultant residue was subjected to silica gel column chromatography. Purification was performed by eluting with mixed liquid of chloroform/methanol/water(=7/3/0.3), and the eluate was concentrated under reduced pressure, to obtain a reaction product (83.0 mg). Identification by ¹H-NMR and ¹³C-NMR confirmed that the product was 2-O-(3′-O-octanoylglyceryl)-6-O-octanoyl ascorbic acid represented by following chemical formula.

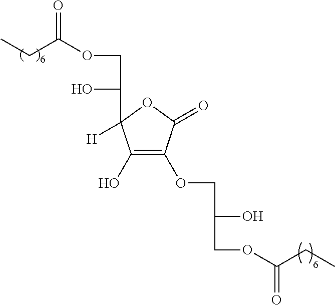

The analyzed results by NMR were as described below. ¹H-NMR (400 MHz, CD₃OD): δ ppm 0.89 (6H, t), 1.30 (16H, brs), 1.59 (4H, m), 2.30 (4H, m), 3.98 (2H, m), 4.06 (2H, m), 4.15 (3H, m), 4.26 (1H, m), 4.76 (1H, d)

¹³C-NMR (100 MHz, CD₃OD): δ ppm 14.4, 23.5, 26.0, 30.0, 30. 1, 32.7, 34.9, 65.4, 65.8, 67.9, 69.3, 74.0, 77.1, 122.0, 161.5, 172.3, 174.8

### Example 5

### Synthesis of 2-O-glyceryl-6-O-dodecanoyl ascorbic acid

Under an argon atmosphere, to 2-O-glyceryl ascorbic acid (50 mg) were added 5 mL of pyridine and n-dodecanoic anhydride (138 mg), and the mixture was stirred for 3 hours at 60° C. Thereafter, ethyl acetate was added and the mixture was extracted with water. The extracted liquid was concentrated under reduced pressure, and 134 mg of the resultant residue was subjected to silica gel column chromatography. Purification was performed by eluting with mixed liquid of chloroform/methanol/water(=7/3/0.3), and the eluate was concentrated under reduced pressure, to obtain a reaction product (73 mg). This reaction product was subjected to mass analysis [ESI, FID (sample direct introduction)] under the following MASS analysis condition 1, to obtain peaks shown below by ESI mass spectrum. Based on the analysis results, it is confirmed that the product was 2-O-glyceryl-6-O-dodecanoyl ascorbic acid represented by following chemical formula.

### [MASS Analysis Condition 1]

Mobile phase: 0.1% formic acid aqueous solution/acetonitrile(=10/90)
Flow rate: 0.2 mL/min
Detector voltage: 1.15 kV
Interface voltage: 4.5 kV
Heat block temperature: 200° C.
Interface temperature: 300° C.
Nebulizer gas flow: 1.5 L/min
Drying gas flow: 15 L/min
Ionization mode: ESI-positive or negative
Measurement mode: Scan mode

### [ESI Mass Spectrum Measurement Result: Detected Peak]

Positive ion: 433 (corresponding to [M+H]⁺),
   455 (corresponding to [M+Na]⁺)
      Negative ion: 432 (corresponding to [M−H]⁻)

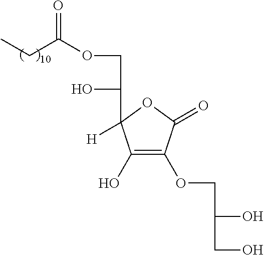

### Example 6

### Synthesis of 2-O-glyceryl-6-O-(2-ethylhexanoyl) ascorbic acid

Under an argon atmosphere, to 2-O-glyceryl ascorbic acid (50 mg) were added 5 mL of pyridine and 2-ethylhexanoic anhydride (118 mg), and the mixture was stirred for 3 hours at 60° C. Thereafter, ethyl acetate was added and the mixture was extracted with water. The extracted liquid was concentrated under reduced pressure, and 134 mg of the resultant residue was subjected to silica gel column chromatography. Purification was performed by eluting with mixed liquid of chloroform/methanol/water(=7/3/0.3), and the eluate was concentrated under reduced pressure, to obtain a reaction product (55 mg). This reaction product was subjected to mass analysis [ESI, FID (sample direct introduction)] under the MASS analysis condition 1 mentioned above, to obtain peaks shown below by ESI mass spectrum. Based on the analysis results, it is confirmed that the product was 2-O-glyceryl-6-O-(2-ethylhexanoyl) ascorbic acid represented by following chemical formula.

### [ESI Mass Spectrum Measurement Result: Detected Peak]

Positive ion: 377 ([M+H]⁺), 399 ([M+Na]⁺)
Negative ion: 375 ([M−H]⁻)

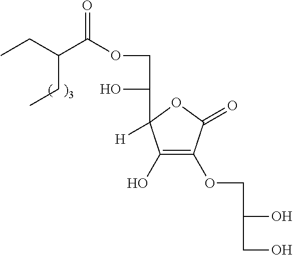

### Example 7

### Synthesis of 2-O-glyceryl-6-O-tetradecanoyl ascorbic acid

To 2-O-glyceryl ascorbic acid (1 g) were added 10 mL of DMSO, triethylamine (0.85 g) and tetradecanoyl chloride (1.48 g), and the mixture was stirred for 1 hour at 50° C. Thereafter, water was added and the mixture was extracted with ethyl acetate. The extracted liquid was concentrated under reduced pressure, and 2.20 g of the resultant residue was subjected to silica gel column chromatography. Purification was performed by eluting with mixed liquid of chloroform/methanol/water (=20/3/0.3̂-6/4/1), and the eluate was concentrated under reduced pressure, to obtain a reaction product (0.35 g). This reaction product was subjected to mass analysis [ESI, FID (sample direct introduction)] under the MASS analysis condition 1 mentioned above, to obtain peaks shown below by

ESI mass spectrum. Based on the analysis results, it is confirmed that the product was 2-O-glyceryl-6-O-tetradecanoyl ascorbic acid represented by following chemical formula.

### [ESI Mass Spectrum Measurement Result: Detected Peak]

Positive ion: 461 ([M+H]⁺), 483 ([M+Na]⁺)
Negative ion: 459 ([M−H]⁻)

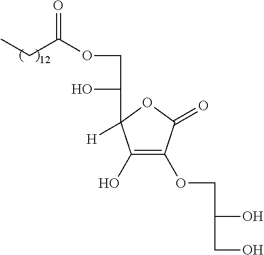

### Example 8

### Synthesis of 2-O-glyceryl-6-O-isostearoyl ascorbic acid

The same procedure as in Example 7 was carried out, except for using isostearoyl chloride (0.97 g) in place of tetradecanoyl chloride (1.48 g) and using triethanol amine (0.95 g) in place of triethylamine (0.85 g) to obtain 0.28 g of a reaction product.

### Example 9

### Synthesis of 2-O-glyceryl-6-O-octadecanoyl ascorbic acid

The same procedure as in Example 7 was carried out, except for using 10 mL of DMF in place of 10 mL of DMSO, using octadecanoyl chloride (1.94 g) in place of tetradecanoyl chloride (1.48 g), using triethylamine (0.40 g) and sodium hydrogen carbonate (0.21 g) in place of triethylamine (0.85 g) and chainging the temperature to 80° C. from 50° C. to obtain 0.36 g of a reaction product.

### Example 10

### Synthesis of 2-O-glyceryl-6-O-oleoyl ascorbic acid

The same procedure as in Example 7 was carried out, except for using 20 mL of concentrated sulfuric acid in place of 10 mL of DMSO and triethylamine (0.85 g), using oleic acid (2.03 g) in place of tetradecanoyl chloride (1.48 g), carrying out the reaction at room temperature for 24 hours (in Example 7; at 50° C. for 1 hour), and conducting washing with saturated sodium bicarbonate aqueous solution after the extraction with ethyl acetate to obtain 0.15 g of a reaction product.

### Example 11

### Synthesis of 2-O-(3′-O-butanoyl glyceryl)-6-O-butanoyl ascorbic acid

The same procedure as in Example 7 was carried out, except for using 10 mL of DMF in place of 10 mL of DMSO, using butanoyl amide (1.74 g) in place of tetradecanoyl chloride (1.48 g) and adding concentrated sulfuric acid (0.02 g) to obtain 0.24 g of a reaction product.

### Example 12

### Synthesis of 2-O-glyceryl-6-O-butanoyl ascorbic acid

The same procedure as in Example 7 was carried out, except for using ethyl butanoate (0.93 g) in place of tetradecanoyl chloride (1.48 g), using concentrated sulfuric acid (0.02 g) in place of triethylamine (0.85 g), adopting the reaction temperature of 80° C. in place of 50° C. to obtain 0.10 g of a reaction product.

### Example 13

### Synthesis of 2-O-(3′-O-butanoyl glyceryl)-3,6-di-O-butanoyl ascorbic acid

The same procedure as in Example 7 was carried out, except for using butanoyl chloride (2.14 g) in place of tetradecanoyl chloride (1.48 g) and using triethylamine (0.80 g) and sodium carbonate (1.27 g) in place of triethylamine (0.85 g) to obtain 0.37 g of a reaction product.

### Example 14

### Synthesis of 2-O-(3′-O-hexadecanoylglyceryl)-3,6-di-O-hexadecanoyl ascorbic acid

The same procedure as in Example 7 was carried out, except for using hexadecanoyl chloride (5.50 g) in place of tetradecanoyl chloride (1.48 g) and using triethylamine (2.83 g) in place of triethylamine (0.85 g) to obtain 0.50 g of a reaction product.

The reaction products obtained in Examples 8-14 were subjected to mass analysis [ESI, FID (sample direct introduction)] under the MASS analysis condition 1 mentioned above (in Example 7). Based on the analysis results, the chemical structures of the reaction products in Examples 8-14 were identified. The chemical structures thus identified and the peaks obtained by ESI mass spectrum are shown in Table 1.

TABLE 1

| | ESI mass spectrum | | |
|---|---|---|---|
| Example | measurement result | | |
| No. | Positive ion | Negative ion | Chemical Structure |
| 8 | 517 ([M + H]+) 539 ([M + Na]+) | 515 ([M − H]−) | 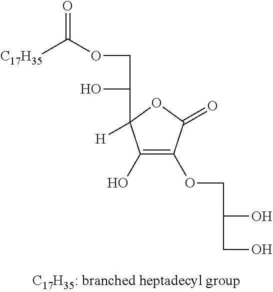 |
| 9 | 517 ([M + H]+) 539 ([M + Na]+) | 515 ([M − H]−) | 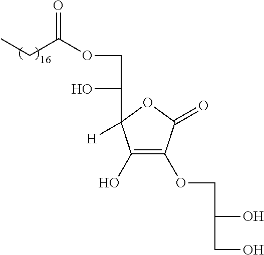 |
| 10 | 515 ([M + H]+) 537 ([M + Na]+) | 513 ([M − H]−) | 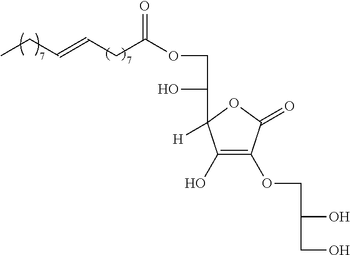 |
| 11 | 391 ([M + H]+) 413 ([M + Na]+) | 389 ([M − H]−) | 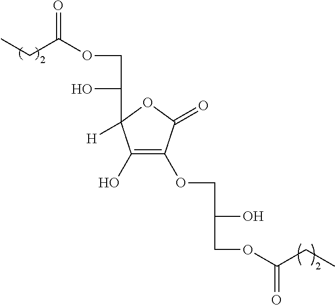 |
| 12 | 321 ([M + H]+) 343 ([M + Na]+) | 319 ([M − H]−) | 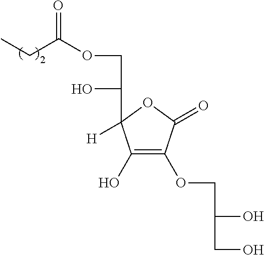 |
| 13 | 461 ([M + H]+) 483 ([M + Na]+) | 459 ([M − H]−) | 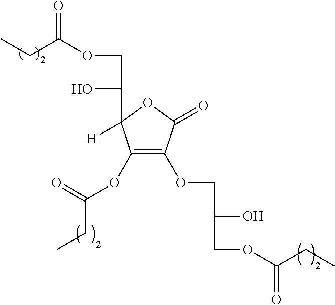 |
| 14 | 988 ([M + Na]+) | — | 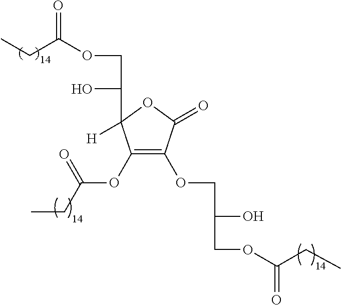 |

### Example 15

### Synthesis of 2-O-(3′-O-octanoylglyceryl)-6-O-butanoyl ascorbic acid

Under an argon atmosphere, to 2-O-glyceryl ascorbic acid (1.00 g) were added 10 mL of DMF, triethylamine (1.01 g) and n-butanoyl chloride (0.34 g), and the mixture was stirred for 1 hour at 50° C. Then, octanoyl chloride (0.65 g) was added to the mixture which was stirred further for 1 hour at 50° C. Thereafter, water was added and the mixture was extracted with ethyl acetate. The extracted liquid was concentrated under reduced pressure, and 2.10 g of the resultant residue was subjected to silica gel column chromatography. Purification was performed by eluting with mixed liquid of chloroform/methanol/water (=20/3/0.3˜6/4/1), and the eluate was concentrated under reduced pressure, to obtain a reaction product (0.34 g). This reaction product was subjected to mass analysis [ESI, FID (sample direct introduction)] under the MASS analysis condition 1 mentioned above, to obtain peaks shown below by ESI mass spectrum. Based on the analysis results, it is confirmed that the product was 2-O-(3′-β-octanoylglyceryl)-6-O-butanoyl ascorbic acid represented by following chemical formula.

### [ESI Mass Spectrum Measurement Result: Detected Peak]

Positive ion: 447 ([M+H]⁺), 469 ([M+Na]⁺)
Negative ion: 445 ([M−H])

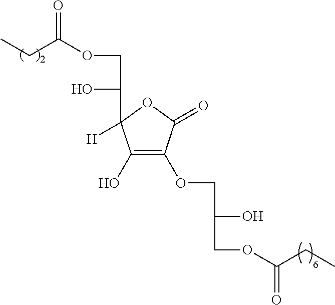

### Example 16

### Synthesis of 2-O-(3′-O-hexadecanoylglyceryl)-6-O-butanoyl ascorbic acid

Under an argon atmosphere, to 2-O-glyceryl ascorbic acid (1.00 g) were added 10 mL of THF, potassium t-butoxide (0.45 g), n-butanoyl chloride (0.34 g) and tetrabutylammonium bromide (0.13 g), and the mixture was stirred for 1 hour at 60° C. Then, hexadecanoyl chloride (1.10 g) was added to the mixture which was stirred further for 1 hour at 50° C. Thereafter, water was added and the mixture was extracted with ethyl acetate. The extracted liquid was concentrated under reduced pressure, and the resultant residue was subjected to silica gel column chromatography. Purification was performed by eluting with mixed liquid of chloroform/methanol/water (=20/3/0.3˜6/4/1), and the eluate was concentrated under reduced pressure, to obtain a reaction product (0.14 g).

### Example 17

### Synthesis of 2-O-(3′-O-butanoylglyceryl)-6-O-dodecanoyl ascorbic acid

The same procedure as in Example 15 was carried out, except for using dodecanoyl chloride (0.70 g) in place of butanoyl chloride (0.34 g) and using butanoyl chloride (0.42 g) in place of octanoyl chloride (0.65 g) to obtain 0.20 g of a reaction product.

### Example 18

### Synthesis of 2-O-glyceryl-3-O-octanoyl ascorbic acid

Under an argon atmosphere, to 2-O-glyceryl ascorbic acid (1.00 g) were added 10 mL of DMSO, triethylamine (0.12 g) and octanoyl chloride (0.65 g), and the mixture was stirred for 5 hour at 25° C. Thereafter, water was added and the mixture was extracted with ethyl acetate. After the extraction, the extracted liquid was washed with saturated saline, then dehydrated with magnesium sulfate, followed by filtration. The filtrate was concentrated under reduced pressure, and 1.95 g of the resultant residue was subjected to silica gel column chromatography. Purification was performed by eluting with mixed liquid of chloroform/methanol/water (=20/3/0.3˜6/4/1), and the eluate was concentrated under reduced pressure, to obtain a reaction product (0.30 g).

### Example 19

### Synthesis of 2-O-glyceryl-3-O-dodecanoyl-6-O-octanoy ascorbic acid

To 2-O-glyceryl ascorbic acid (1.00 g) were added 10 mL of DMF, triethylamine (0.12 g) and dodecanoyl chloride (0.70 g), and the mixture was stirred for 5 hours at 50° C. Then, triethylamine (0.48 g) and octanoyl anhydride (1.30 g) were added to the mixture which was stirred further for 5 hours at 50° C. Thereafter, water was added and the mixture was extracted with ethyl acetate. After the extraction, the extracted liquid was washed with saturated saline, then dehydrated with magnesium sulfate, followed by filtration. The filtrate was concentrated under reduced pressure, and 3.25 g of the resultant residue was subjected to silica gel column chromatography. Purification was performed by eluting with mixed liquid of chloroform/methanol/water (=20/310.3˜6/4/1), and the eluate was concentrated under reduced pressure, to obtain a reaction product (0.45 g).

### Example 20

### Synthesis of 3-O-glyceryl-6-O-butanoyl ascorbic acid

The same procedure as in Example 7 was carried out, except for using 3-O-glyceryl ascorbic acid (1.00 g) in place of 2-O-glyceryl ascorbic acid (1.00 g) and using butanoyl chloride (0.42 g) in place of octanoyl chloride (0.65 g) to obtain 0.25 g of a reaction product.

### Example 21

### Synthesis of 3-O-glyceryl-6-O-octanoyl ascorbic acid

The same procedure as in Example 7 was carried out, except for using 3-O-glyceryl ascorbic acid (1.00 g) in place of 2-O-glyceryl ascorbic acid (1.00 g), using 10 mL of DMF in place of 10 mL of DMSO and using octanoyl anhydride (1.08 g) in place of octanoyl chloride (0.65 g) to obtain 0.30 g of a reaction product.

### Example 22

### Synthesis of 3-O-glyceryl-6-O-hexadecanoyl ascorbic acid

The same procedure as in Example 7 was carried out, except for using 3-O-glyceryl ascorbic acid (1.00 g) in place of 2-O-glyceryl ascorbic acid (1.00 g), using 10 mL of pyridine in place of 10 mL of DMSO, using hexadecanoyl anhydride (1.78 g) in place of octanoyl chloride (0.65 g) and not using triethyl amine (0.85 g) to obtain 0.40 g of a reaction product.

### Example 23

### Synthesis of 3-O-(3′-O-dodecanoylglyceryl)-6-O-butanoyl ascorbic acid

The same procedure as in Example 7 was carried out, except for using 3-O-glyceryl ascorbic acid (1.00 g) in place of 2-O-glyceryl ascorbic acid (1.00 g) and using dodecanoyl anhydride (1.64 g) in place of octanoyl chloride (0.65 g) to obtain 0.42 g of a reaction product.

The reaction products obtained in Examples 16-23 were subjected to mass analysis [ESI, FID] under the MASS analysis condition 1 mentioned above (in Example 7). Based on the analysis results, the chemical structures of the reaction products in Examples 16-23 were identified. The chemical structures thus identified and the peaks obtained by ESI mass spectrum are shown in Table 2.

TABLE 2

| | ESI mass spectrum | | |
|---|---|---|---|
| Example | measurement result | | |
| No. | Positive ion | Negative ion | Chemical Structure |
| 16 | 559 ([M + H]+) 581 ([M + Na]+) | 557 ([M − H]−) | 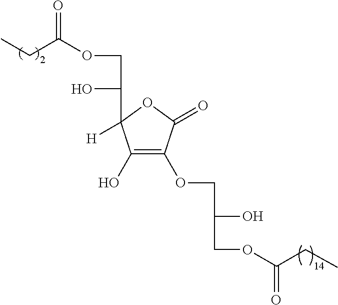 |
| 17 | 503 ([M + H]+) 525 ([M + Na]+) | 501 ([M − H]−) | 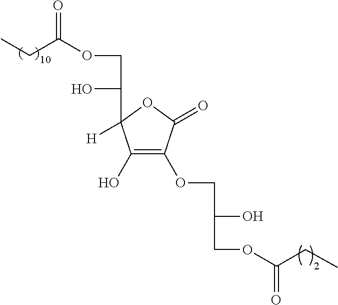 |
| 18 | 377 ([M + H]+) 399 ([M + Na]+) | 375 ([M − H]−) | 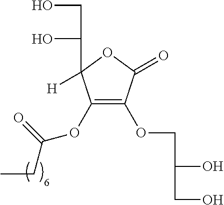 |
| 19 | 559 ([M + H]+) 581 ([M + Na]+) | 557 ([M − H]−) | 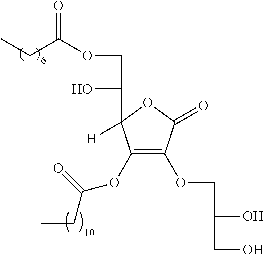 |
| 20 | 321 ([M + H]+) 343 ([M + Na]+) | 319 ([M − H]−) | 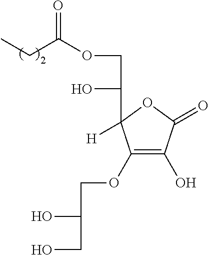 |
| 21 | 337 ([M + H]+) 399 ([M + Na]+) | 375 ([M − H]−) | 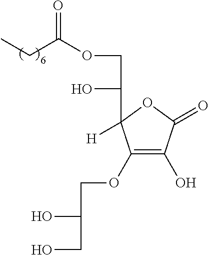 |
| 22 | 489 ([M + H]+) 511 ([M + Na]+) | 487 ([M − H]−) | 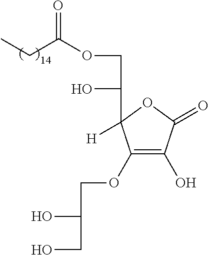 |
| 23 | 503 ([M + H]+) 525 ([M + Na]+) | 501 ([M − H]−) | 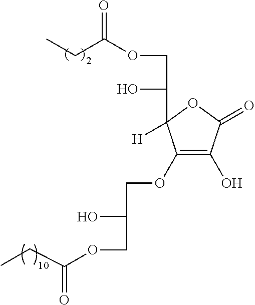 |

### Test Example 1

### Melanin Production Suppressing Evaluation Test

As a test for a whitening effect, evaluation of the action of B16 melanoma 4A5 cell on theophylline-induced melanine production was carried out on a glyceryl ascorbic acid acylated derivative of the present invention, according to the following procedure. The same evaluations were carried out also on arbutin, ascorbic acid, sodium ascorbate, magnesium ascorbyl phosphate, glucosyl ascorbic acid, and 3-O-glyceryl ascorbic acid and 2-O-glyceryl ascorbic acid obtained in the Synthesis Examples mentioned above, as comparison.

(1) B16 mouse melanoma 4A5 strain was sowed onto a 48-well plate at a cell density of 2.0×10⁴ cells/well.
(2) Culturing was performed for 24 hours with Dulbecco's Modified Eagle's medium (manufactured by SIGMA. Hereinafter, abbreviated as D-MEM) containing 10% fetal bovine serum (manufactured by Roshe Diagnostics). Then, it was changed to 0.2 mM theophylline and a 10% fetal bovine serum-containing D-MEM which contains a sample of given concentration.
(3) After culturing for 3 days in the co-existence of a sample, the medium was removed using an aspirator. Then, after distilled water was added, cells were broken by an ultrasonic wave.
(4) Thereafter, the amount of protein was determined using BCA protein assay kit (manufactured by PIERCE), and the produced amount of melanine was measured by an alkali solubilizing method described later. To the cell-destructed solution was added sodium hydroxide so as to give a final concentration of 2 mol/L and the mixture was dissolved by heating (60° C., 15 minutes), then, the absorbance at 450 nm was measured using a micro plate reader. The melanine amount was calculated from a calibration curve made using synthetic melanine (SIGMA) as a standard. The melanine amount per unit protein was calculated by dividing the melanine amount by the protein amount.
(5) The melanine production suppressing rate was calculated according to the following formula.

Melanine production suppressing rate(%)=[1−(A−B)/(C−B)]×100

[wherein, A represents the melanine amount per unit protein (g/g) in adding a sample, B represents the melanine amount per unit protein (g/g) in the normal group, and C represents the melanine amount per unit protein (g/g) in the control group.]

The normal group means the case where neither theophylline nor sample was added.(that is; theophylline(−) and sample(−))

The control group means the case where neither sample was not added but theophylline was added.(that is; theophylline (+) and sample(−))

In the following Tables, the melanine production suppressing rate in applying a sample at a concentration of 100 μl or less is expressed as described below.

Less than 30%: Δ

30 to 49%: ◯

50% or more: ⊚

TABLE 3

| Example | | Whitening |
|---|---|---|
| No. | Sample | effect |
| Comparison | Arbutin | ⊚ |
| Comparison | Ascorbic acid | Δ |
| Comparison | Sodium ascorbate | Δ |
| Comparison | Magnesium ascorbyl phosphate | Δ |
| Comparison | Glucosyl ascorbic acid | Δ |
| Comparison | 3-O-glyceryl ascorbic acid | Δ |
| Comparison | 2-O-glyceryl ascorbic acid | Δ |
| 1 | 2-O-glyceryl-6-O-butanoyl ascorbic acid | ◯ |
| 2 | 2-O-glyceryl-6-O-octanoyl ascorbic acid | ⊚ |
| 3 | 2-O-glyceryl-6-O-hexadecanoyl ascorbic acid | ⊚ |
| 4 | 2-O-(3′-O-octanoylglyceryl)-6-O-octanoyl | ⊚ |
| | ascorbic acid | |
| 5 | 2-O-glyceryl-6-O-dodecanoyl ascorbic acid | ⊚ |
| 6 | 2-O-glyceryl-6-O-(2-ethylhexanoyl)-ascorbic | ⊚ |
| | acid | |
| 7 | 2-O-glyceryl-6-O-tetradecanoyl ascorbic acid | ⊚ |
| 8 | 2-O-glyceryl-6-O-isostearoyl ascorbic acid | ⊚ |
| 9 | 2-O-glyceryl-6-O-octadecanoyl ascorbic acid | ⊚ |
| 10 | 2-O-glyceryl-6-O-oleoyl ascorbic acid | ⊚ |
| 11 | 2-O-(3′-O-butanoylglyceryl)-6-O-butanoyl | ◯ |
| | ascorbic acid | |
| 13 | 2-O-(3′-O-butanoylglyceryl)-3,6-di-O-butanoyl | ⊚ |
| | ascorbic acid | |
| 16 | 2-O-(3′-O-hexadecanoylglyceryl)-6-O-butanoyl | ⊚ |
| | ascorbic acid | |
| 19 | 2-O-glyceryl-3-O-dodecanoyl-6-O- | ⊚ |
| | octadecanoyl ascorbic acid | |
| 21 | 3-O-glyceryl-6-O-octanoyl ascorbic acid | ⊚ |

The results in Table 3 shows that the glyceryl ascorbic acid acylated derivative of the present invention has a whitening effect higher than the effect of ascorbic acid, sodium ascorbate, known ascorbic acid derivatives, namely, glucosyl ascorbic acid and magnesium ascorbyl phosphate, 3-O-glyceryl ascorbic acid and 2-O-glyceryl ascorbic acid.

### Test Example 2

### Collagen Production Evaluation Test

The collagen production promoting effects of the glyceryl ascorbic acid acylated derivatives of the present invention were evaluated, according to the following procedure. As comparison, arbutin, ascorbic acid, sodium ascorbate, magnesium ascorbyl phosphate, glucosyl ascorbic acid, 3-O-glyceryl ascorbic acid and 2-O-glyceryl ascorbic acid were used like in Test Example 1.

Normal human dermal fibroblasts were regulated with a 10% (v/v) fetal bovine serum (manufactured by Invitrogen)-containing D-MEM so as to give a cell density of 2.5×10⁴ cells/well, then, pre-incubation for 24 hours was performed on a 96-well plate. After the medium was removed, a sample regulated with a 5% (v/v) fetal bovine serum-containing D-MEM to a concentration of 100 μM was added to each well, then, cultured for 48 hours at 37° C. and 5% CO₂. After completion of culturing, the amount of free collagen was measured using Sircol collagen assay kit (manufactured by Biocolor).

The collagen production amount in applying a sample at a concentration of 100 μM is obtained as % value when the control group is 100%, and the results are shown in Table 4 based on the following standard.

<100%: ±

100 to 200%: +

TABLE 4

| | | Collagen |
|---|---|---|
| | | production |
| Example | | promoting |
| No. | Sample | effect |
| Comparison | Arbutin | ± |
| Comparison | Ascorbic acid | + |
| Comparison | Sodium ascorbate | + |
| Comparison | Magnesium ascorbyl phosphate | + |
| Comparison | Glucosyl ascorbic acid | + |
| Comparison | 3-O-glyceryl ascorbic acid | + |
| Comparison | 2-O-glyceryl ascorbic acid | + |
| 1 | 2-O-glyceryl-6-O-butanoyl ascorbic acid | + |
| 2 | 2-O-glyceryl-6-O-octanoyl ascorbic acid | + |
| 3 | 2-O-glyceryl-6-O-hexadecanoyl ascorbic acid | + |
| 4 | 2-O-(3′-O-octanoylglyceryl)-6-O-octanoyl | + |
| | ascorbic acid | |
| 5 | 2-O-glyceryl-6-O-dodecanoyl ascorbic acid | + |
| 6 | 2-O-glyceryl-6-O-(2-ethylhexanoyl)-ascorbic | + |
| | acid | |
| 13 | 2-O-(3′-O-butanoyl glyceryl)-3,6-di-O-butanoyl | + |
| | ascorbic acid | |
| 15 | 2-O-(3′-O-octanoyl glyceryl)-6-O-butanoyl | + |
| | ascorbic acid | |
| 17 | 2-O-(3′-O-butanoyl glyceryl)-6-O-dodecanoyl | + |
| | ascorbic acid | |
| 18 | 2-O-glyceryl-3-O-octanoyl ascorbic acid | + |
| 20 | 3-O-glyceryl-6-O-butanoyl ascorbic acid | + |
| 22 | 3-O-glyceryl-6-O-hexadecanoyl ascorbic acid | + |
| 23 | 3-O-(3′-O-dodecanoyl glyceryl)-6-O-butanoyl | + |
| | ascorbic acid | |

The evaluation results in Table 4 show that the glyceryl ascorbic acid acylated derivative of the present invention has a collagen production promoting effect which is approximately the same as that of ascorbic acid and known ascorbic acid derivatives such as magnesium ascorbyl phosphate, glucosyl ascorbic acid and the like, which are known as substances having particularly excellent collagen production promoting effect.

### Test Example 3

### Stability Test

Using 2-O-glyceryl-6-O-octanoyl ascorbic acid obtained in Example 2 and 2-O-glyceryl-6-O-octadecanoyl ascorbic acid obtained in Example 9, an evaluation was made on the stability of odor and color change when stored at 50° C. for 4 weeks, according to the following procedure. For comparison, magnesium ascorbyl phosphate, ascorbic acid and 3-O-glyceryl ascorbic acid were used.

Aqueous solutions (2%) of various test samples were adjusted to pH7 with a dilute sodium hydroxide aqueous solution or a dilute hydrochloric acid aqueous solution, and charged into 50 mL screw tubes. The tubes were sealed and stored at 50° C. for 4 weeks. The odor and degree of color change immediately after preparation, after storage for two weeks and after storage for four weeks were evaluated by ten panelists based on the following methods and according to the following criterion. The results are shown in Table 5.

### [Odor]

3: almost no odor

2: slight abnormal odor

1: intense abnormal odor

### [Color Change]

3: almost no change as compared with that immediately after preparation

2: color change is observed as compared with that immediately after preparation

1: intense color change is observed as compared with that immediately after preparation

Based on the evaluation results, classification was conducted as shown below.

◯: total point of 10 panelists is 25 or more

Δ: total point of 10 panelists is 16 to 24

x: total point of 10 panelists is 15 or less

TABLE 5

| Storage period | | 0 | 2 weeks | 4 weeks |
|---|---|---|---|---|
| Magnesium ascorbyl | Odor | ◯ | ◯ | ◯ |
| phosphate | Color change | ◯ | Δ | X |
| (for Comparison) | | | | |
| Ascorbic acid | Odor | ◯ | Δ | X |
| (for Comparison) | Color change | ◯ | X | X |
| 3-O-glyceryl ascorbic | Odor | ◯ | ◯ | ◯ |
| acid(for Comparison) | Color change | ◯ | ◯ | Δ |
| 2-O-glyceryl-6-O-octanoyl | Odor | ◯ | ◯ | ◯ |
| ascorbic acid | Color change | ◯ | ◯ | ◯ |
| (Example 2) | | | | |
| 2-O-glyceryl-6-O- | Odor | ◯ | ◯ | ◯ |
| octadecanoyl ascorbic | Color change | ◯ | ◯ | ◯ |
| acid (Example 9) | | | | |

Based on the evaluation results in Table 5, it was confirmed that the 2-O-glyceryl ascorbic acid acylated derivative of the present invention was excellent in stability regarding odor and color change.

### Test Example 4

### Emulsion Stability Test

Emulsion compositions having formulations shown in Table 6 were prepared using 2-O-glyceryl-6-O-hexadecanoyl ascorbic acid of Example 3, 2-O-(3′-O-butanoylglyceryl)-3,6-di-O-butanoyl ascorbic acid of Example 13, and, as comparisons, ascorbic acid, magnesium ascorbyl phosphate and 2-O-glyceryl ascorbic acid, respectively. The prepared compositions were charged in 50 mL screw tubes which were then sealed, and stored at 50° C. for 1 week. One week after, 10 panelists were allowed to evaluate the appearance of the emulsion composition according to the following criterion (evaluation standard).

3: Separation of aqueous layer and oil layer is not observed

2: Aqueous layer is slightly floating and standing out

1: Separation of aqueous layer and oil layer is observed

Based on the evaluation results, classification was set as described below. The results are shown in Table 6.

◯: Total point of 10 panelists is 25 or more

Δ: Total point of 10 panelists is 16 to 24

x: Total point of 10 panelists is 15 or less

TABLE 6

| | Comparison | | | Example | |
|---|---|---|---|---|---|
| | Test 1 | Test 2 | Test 3 | Test 4 | Test 5 |
| Ascorbic acid | 5.00 | — | — | — | — |
| Magnesium ascorbyl | — | 5.00 | — | — | — |
| phosphate | | | | | |
| 3-O-glyceryl ascorbic | — | — | 5.00 | — | — |
| acid | | | | | |
| 2-O-glyceryl-6-O- | — | — | — | 5.00 | — |
| hexadecanoyl ascorbic | | | | | |
| acid (Example 3) | | | | | |
| 2-O-(3′-O-butanoyl | — | — | — | — | 5.00 |
| glyceryl)-3,6-di-O- | | | | | |
| butanoyl ascorbic acid | | | | | |
| (Example 13) | | | | | |
| Cetearyl alcohol | 0.80 | 0.80 | 0.80 | 0.80 | 0.80 |
| Cetearyl glucoside | 0.20 | 0.20 | 0.20 | 0.20 | 0.20 |
| 1,3-butylene glycol | 5.00 | 5.00 | 5.00 | 5.00 | 5.00 |
| POE(15) lauryl alcohol | 0.50 | 0.50 | 0.50 | 0.50 | 0.50 |
| ether | | | | | |
| Ethanol | 5.00 | 5.00 | 5.00 | 5.00 | 5.00 |
| Citric acid | 0.01 | 0.01 | 0.01 | 0.01 | 0.01 |
| Sodium citrate | 0.02 | 0.02 | 0.02 | 0.02 | 0.02 |
| Potassium hydroxide | suitable | suitable | suitable | suitable | suitable |
| | amount | amount | amount | amount | amount |
| Water | residual | residual | residual | residual | residual |
| | amount | amount | amount | amount | amount |
| Emulsion stability | X | X | Δ | ◯ | ◯ |

In Table 6, formulation was shown by mass parts, and the residual amount means an amount necessary for adjusting the total compounding amount to 100 parts by mass. The same shall apply also to residual amount in Tables described later.

As shown in Table 6, it was confirmed that the glyceryl ascorbic acid acylated derivative of the present invention was excellent in emulsion stability.

### Test Example 5

### Skin Permeability Test

Permeability of the glyceryl ascorbic acid acylated derivatives of Examples 1 to 6 into skin was tested by the following method. For comparison, sodium ascorbate, magnesium ascorbyl phosphate, glucosyl ascorbic acid, 3-O-glyceryl ascorbic acid, 2-O-glyceryl ascorbic acid and 3-O-cetyl ascorbic acid were used.

Each test sample (3 g) was dissolved in 100 mL of water, and pH thereof was adjusted to 7 with a dilute sodium hydroxide aqueous solution or a dilute hydrochloric acid aqueous solution to prepare a sample solution. A paper filter of 2 cm×2 cm was placed on a forearm portion and 0.5 mL of the sample solution was applied on this, and covered and fixed by a polyamide film. After 8 hours, the sample-applied portion on forearm was washed, and tape stripping was carried out 15 times. From each tape, the sample was extracted with a mixed solvent of water/acetone (=1/1), and the extraction solution was analyzed using HPLC (high performance liquid chromatography) at a detection wavelength of 254 nm, and the degree of permeation of the sample into skin was checked. The permeability of the sample into skin was judged according to the following criterion (evaluation standard). The results are shown in Table 7.

### (Judgment Criterion)

◯: Sample is detected until 10 to 15 times of tape stripping

Δ: Sample is detected until 3 to 9 times of tape stripping

x: Sample is detected until 0 to 2 times of tape stripping, or sample is not detected

TABLE 7

| | Sample | Permeability |
|---|---|---|
| Comparison | Sodium ascorbate | X |
| Comparison | Magnesium ascorbyl phosphate | X |
| Comparison | Glucosyl ascorbic acid | X |
| Comparison | 3-O-glyceryl ascorbic acid | X |
| Comparison | 2-O-glyceryl ascorbic acid | Δ |
| Comparison | 3-O-cetylglyceryl ascorbic acid | ◯ |
| Example 1 | 2-O-glyceryl-6-O-butanoyl ascorbic acid | ◯ |
| Example 2 | 2-O-glyceryl-6-O-octanoyl ascorbic acid | ◯ |
| Example 3 | 2-O-glyceryl-6-O-hexadecanoyl | ◯ |
| | ascorbic acid | |
| Example 4 | 2-O-(3′-O-octanoylglyceryl-)-6-O- | ◯ |
| | octanoyl ascorbic acid | |
| Example 5 | 2-O-glyceryl-6-O-dodecanoyl | ◯ |
| | ascorbic acid | |
| Example 6 | 2-O-glyceryl-6-O-(2-ethylhexanoyl) | ◯ |
| | ascorbic acid | |

### Test Example 6

### Moisturizing Test

An in vitro moisturizing test and an in vivo moisturizing test of the glyceryl ascorbic acid acylated derivatives of Examples 1 to 6 were carried out according to the following manner. For comparison, sodium ascorbate, magnesium ascorbyl phosphate, glucosyl ascorbic acid, 3-O-glyceryl ascorbic acid, 2-O-glyceryl ascorbic acid and 3-O-cetyl ascorbic acid were used.

### 1. In Vitro Moisturizing Test

A sample shown later was dried, and the dried sample was spread in an amount of about 0.4 g (this weight is represented by W0) on the bottom of a weighing bottle (diameter: 3.6 cm, height excluding lid: 1.8 cm) so as to give uniform thickness. The sample contained in the weighing bottle was allowed to stand still in a constant humidity and constant temperature vessel (ENVIROS KCL-1000, EYELA) under environments of 25° C. and 65% RH. The weight was measured periodically, waiting until sufficient moisture absorption to attain equilibrium of weight increase (about 48 hours). Thereafter, the sample was transferred to under environments of 25° C. and 20% RH (in sealed vessel containing a saturated CH₃COOK aqueous solution filled in the bottom part). From the weight after 24 hours (this weight is represented by W1), the amount of retained water per 1 g of the dried sample was calculated according to the following formula.

(W1−W0)/W0

From the water amount thus calculated, the moisturizing effect was judged based on the following judging criterion, and the results are shown in Table 8.

### (Judging Criterion)

◯: 35 mg or more

Δ: 15 mg or more and less than 35 mg

x: less than 15 mg

### 2. In Vivo Moisturizing Test

Each test sample (5 g) was dissolved in 100 mL of water, and pH thereof was adjusted to 7 with a dilute sodium hydroxide aqueous solution or a dilute hydrochloric acid aqueous solution to prepare a sample solution. A paper filter of 1 cm×1 cm was placed on a forearm portion and 0.3 mL of the prepared sample solution was applied on this, and covered and fixed by a polyamide film. Then, 3 hours and 8 hours after, the stratum corneum water content at the sample-applied portion was measured using CORNEOMETER CM825 (manufactured by Integral Corporation). The test was carried out with 10 subjects, and evaluated according to the following criterion (evaluation standard).

### (Evaluation Criterion)

3: Stratum corneum water content increased by 15% or more, based on the water content before sample application

2: Stratum corneum water content increased by 0 to less than 15%, based on the water content before sample application

1: There was no change or there was a reduction in stratum corneum water content, before and after sample application.

Based on the evaluation results, classification was set as described below. The results are shown in Table 8.

◯: Total points of 10 subjects is 25 or more

Δ: Total points of 10 subjects is 16 to 24

x: Total points of 10 subjects is 15 or less

TABLE 8

| | | in | in vivo after | |
|---|---|---|---|---|
| | Sample | vitro | 3 hours | 8 hours |
| Comparison | Sodium ascorbate | X | X | X |
| Comparison | Magnesium ascorbyl phosphate | X | X | X |
| Comparison | Glucosyl ascorbic acid | Δ | X | X |
| Comparison | 3-O-glyceryl ascorbic acid | ◯ | X | Δ |
| Comparison | 2-O-glyceryl ascorbic acid | ◯ | Δ | Δ |
| Comparison | 3-O-cetylglyceryl ascorbic acid | X | X | X |
| Example 1 | 2-O-glyceryl-6-O-butanoyl | X | ◯ | ◯ |
| | ascorbic acid | | | |
| Example 2 | 2-O-glyceryl-6-O-octanoyl | X | ◯ | ◯ |
| | ascorbic acid | | | |
| Example 3 | 2-O-glyceryl-6-O-hexadecanoyl | X | ◯ | ◯ |
| | ascorbic acid | | | |
| Example 4 | 2-O-(3′-octanoylglyceryl-)-6-O- | X | ◯ | ◯ |
| | octanoyl ascorbic acid | | | |
| Example 5 | 2-O-glyceryl-6-O-dodecanoyl | X | ◯ | ◯ |
| | ascorbic acid | | | |
| Example 6 | 2-O-glyceryl-6-O(2-ethyl | X | ◯ | ◯ |
| | hexanoyl) ascorbic acid | | | |

As shown in Table 8, the glyceryl ascorbic acid acylated derivative of the present invention is excellent in a moisturizing property when applied on skin, as compared with other ascorbic acid and derivatives thereof. The glyceryl ascorbic acid acylated derivative is endowed with hydrophobicity and excellent also in permeability into skin, since the stratum corneum water content increased in a shorter period of time as compared with glyceryl ascorbic acid. Further, in comparison with 3-O-cetylglyceryl ascorbic acid which is excellent in solubility in an oil and excellent in permeability into skin, a high moisturizing effect is shown in the in vivo test, and a moisturizing effect ascribable to glyceryl ascorbic acid showing an in vitro moisturizing property is obtained.

### Example 24

### Cream

Oil phase raw materials (1) to (5) and aqueous phase raw materials (6) to (10) of a formulation shown in Table 9 were heated up to 70° C. and dissolved, to prepare an oil phase and an aqueous phase, respectively. Thereafter, the oil phase was added to the aqueous phase and the mixture was pre-emulsified, then emulsified uniformly by Homo Mixer, followed by cooling down to room temperature while thoroughly stirring, to prepare a cream. This cream is excellent in sensory texture upon use, a moisturizing effect and a whitening effect since it contains a glyceryl ascorbic acid acylated derivative of the present invention, and the cream is believed to be used suitably as skin cosmetics. The compounding amount in the following Tables denotes mass %.

TABLE 9

| | | Compounding |
|---|---|---|
| No | Name of component | amount (%) |
| 1 | Squalane | 8.0 |
| 2 | Vaseline | 5.0 |
| 3 | Stearyl alcohol | 5.0 |
| 4 | Polyoxyethylene(25) cetyl ether | 2.5 |
| 5 | Glyceryl monostearate | 1.5 |
| 6 | 2-O-glyceryl-6-O-butanoyl ascorbic | 3.0 |
| | acid (produced in Example 1) | |
| 7 | Glycerin | 5.0 |
| 8 | preservative | suitable amount |
| 9 | pH regulator | suitable amount |
| 10 | purified water | residual amount |

### Example 25

### Milky Lotion

Oil phase part raw materials (1) to (9) and aqueous phase part raw materials (10) to (13) having compositions shown in Table 10 were heated up to 70° C. and dissolved, to prepare an oil phase and an aqueous phase, respectively. Thereafter, the oil phase was added to the aqueous phase. The mixture is pre-emulsified, then emulsified uniformly by a homo-mixer, followed by cooling down to room temperature while stirring thoroughly, to prepare a milky lotion. This milky lotion is excellent in a feeling of use, a moisturizing effect and a whitening effect since it contains a glyceryl ascorbic acid acylated derivative of the present invention, and the milky lotion is believed to be used suitably as a skin cosmetic.

TABLE 10

| | | Compounding |
|---|---|---|
| No | Name of component | amount |
| 1 | Isosteary palmitate | 4.0 |
| 2 | Jojoba oil | 2.0 |
| 3 | Dimethylpolysiloxane | 2.0 |
| 4 | Cetanol | 1.0 |
| 5 | Stearic acid | 1.5 |
| 6 | Bees wax | 2.5 |
| 7 | Paraffin wax | 2.5 |
| 8 | Polyoxyethylene(20) sorbitan | 1.2 |
| | monostearate | |
| 9 | Polyoxyethylene(40) sorbitol | 1.5 |
| | tetraoleate | |
| 10 | Propylene glycol | 10.0 |
| 11 | 2-O-glyceryl-6-O-octanoyl ascorbic | 3.0 |
| | acid (produced in Example 2) | |
| 12 | Antiseptic | suitable amount |
| 13 | Purified water | residual amount |

### Example 26

### Milky Lotion

Oil phase part raw materials (4) to (10) and aqueous phase part raw materials (1) to (3) and (11) to (12) having compositions shown in Table 11 were heated up to 70° C. and dissolved, to prepare an oil phase and an aqueous phase, respectively. Thereafter, the oil phase was added to the aqueous phase. The mixture is pre-emulsified, then emulsified uniformly by a homo-mixer, followed by cooling down to room temperature while stirring thoroughly, to prepare a milky lotion. This milky lotion is excellent in a feeling of use, a moisturizing effect and a whitening effect since it contains a glyceryl ascorbic acid acylated derivative of the present invention, and the milky lotion is believed to be used suitably as skin cosmetics.

TABLE 11

| | | Compounding |
|---|---|---|
| No | Name of component | amount |
| 1 | Dipropylene glycol | 3.0 |
| 2 | Sorbitansesqui oleate | 3.0 |
| 3 | Polyoxyethylene(20) sorbitan monooleate | 1.0 |
| 4 | 2-O-glyceryl-6-O-dodecanoyl ascorbic | 3.0 |
| | acid (produced in Example 5) | |
| 5 | Micro crystalline wax | 1.5 |
| 6 | Bees wax | 2.5 |
| 7 | Lanolin | 2.0 |
| 8 | Liquid paraffin | 16.5 |
| 9 | Squalane | 10.0 |
| 10 | Perfume | suitable amount |
| 11 | Antiseptic | suitable amount |
| 12 | Purified water | residual amount |

### Example 27

### Cream

Oil phase part raw materials (1) to (3) and aqueous phase part raw materials (4) to (10) having compositions shown in Table 12 were heated up to 70° C. and dissolved, to prepare an oil phase and an aqueous phase, respectively. Thereafter, the oil phase was added to the aqueous phase. The mixture was pre-emulsified, then emulsified uniformly by Homo Mixer, followed by cooling down to room temperature while thoroughly stirring, to prepare a cream. This cream is excellent in a feeling of use, a moisturizing effect and a whitening effect since it contains a glyceryl ascorbic acid acylated derivative of the present invention, and the cream is believed to be used suitably as a skin cosmetic.

TABLE 12

| | | Compounding |
|---|---|---|
| No | Name of component | amount |
| 1 | Liquid paraffin | 15.0 |
| 2 | Vaseline | 15.0 |
| 3 | 2-O-glyceryl-6-O-hexadecanoyl ascorbic | 5.0 |
| | acid (produced in Example 3) | |
| 4 | Carboxyvinylpolymer | 0.1 |
| 5 | Xanthan gum | 0.1 |
| 6 | Hardened castor oil | 3.0 |
| | polyoxyethylene(40) derivative | |
| 7 | Sodium hydroxide | 0.05 |
| 8 | Perfume | suitable amount |
| 9 | Antiseptic | suitable amount |
| 10 | Purified water | residual amount |

### Example 28

### Cream

Oil phase part raw materials (1) to (5) and aqueous phase part raw materials (6) to (10) having compositions shown in Table 13 were heated up to 70° C. and dissolved, to prepare an oil phase and an aqueous phase, respectively. Thereafter, the oil phase was added to the aqueous phase. The mixture was pre-emulsified, then emulsified uniformly by Homo Mixer, followed by cooling down to room temperature while thoroughly stirring, to prepare a cream. This cream is excellent in a feeling of use, a moisturizing effect and a whitening effect since it contains a glyceryl ascorbic acid acylated derivative of the present invention, and the cream is believed to be used suitably as a skin cosmetic.

TABLE 13

| | | Compounding |
|---|---|---|
| No | Name of component | amount |
| 1 | Cetyl alcohol | 2.0 |
| 2 | Stearyl alcohol | 3.0 |
| 3 | Squalane | 6.5 |
| 4 | Glyceryl tri-2-ethylhexanoate | 5.5 |
| 5 | Methylpolysiloxane | 5.5 |
| 6 | 2-O-glyceryl-6-O-(2-ethylhexanoyl) | 4.0 |
| | ascorbic acid (produced in Example 6) | |
| 7 | 1,3-butylene glycol | 5.0 |
| 8 | Hydroxyethylcellulose | 0.2 |
| 9 | preservative | suitable amount |
| 10 | purified water | residual amount |

### Example 29

### Lotion

Raw materials (1) to (6) having compositions shown in Table 14 were mixed while thoroughly stirring, to prepare a lotion. This lotion is excellent in a feeling of use, a moisturizing effect and a whitening effect since it contains a glyceryl ascorbic acid acylated derivative of the present invention, and the lotion is believed to be used suitably as a skin cosmetic.

TABLE 14

| | | Compounding |
|---|---|---|
| No | Name of component | amount |
| 1 | 2-O-glyceryl-6-O-butanoyl ascorbic acid | 7.0 |
| | (produced in Example 1) | |
| 2 | Alcohol | 8.0 |
| 3 | Citric acid | 0.01 |
| 4 | Sodium Citrate | 0.015 |
| 5 | Potassium Glycyrrhizinate | 0.03 |
| 6 | Purified water | residual amount |

1. A glyceryl ascorbic acid acylated derivative represented by the following general formula (I): 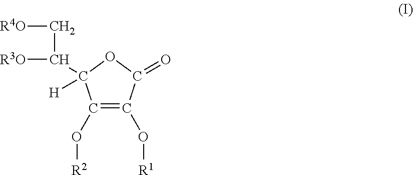
wherein, R¹ and R² represent a hydrogen atom, R⁵—O—CH₂—CH(OH)—CH₂— or an acyl group represented by R⁶—CO—, R³ and R⁴ represent a hydrogen atom or a group represented by R⁵ wherein R⁵ represents a hydrogen atom or an acyl group represented by R⁶—CO—, and R⁶ represents a hydrogen atom, an alkyl group having 1 to 22 carbon atoms or an alkenyl group having 2 to 22 carbon atoms, provided that, at least one of R¹ and R² is a group represented by R⁵—O—CH₂—CH(OH)—CH₂—, and when both R³ and R⁴ are a hydrogen atom, at least one of R¹ and R² is a group represented by R⁶—CO—O—CH₂—CH(OH)—CH₂—.

2. A glyceryl ascorbic acid acylated derivative or its salt according to claim 1, in which R¹ and R² represent a hydrogen atom or R⁵—O—CH₂—CH(OH)—CH₂—, R³ represents a hydrogen atom and R⁴ represents a hydrogen atom or an acyl group represented by R⁶—CO—.

3. A glyceryl ascorbic acid acylated derivative or its salt according to claim 2, in which only one of R¹, R² and R⁴ represents R⁶—CO—.

4. A glyceryl ascorbic acid acylated derivative or its salt according to claim 1, in which R¹ and R² represent a hydrogen atom or R⁵—O—CH₂—CH(OH)—CH₂—, R³ and R⁵ represent a hydrogen atom, and R⁴ represents R⁶—CO—.

5. A glyceryl ascorbic acid acylated derivative or its salt according to claim 1, in which R¹ represents R⁵—O—CH₂—CH(OH)—CH₂—, R², R³ and R⁵ represent a hydrogen atom, and R⁴ represents R⁶—CO—.

6. A glyceryl ascorbic acid acylated derivative or its salt according to claim 5, in which R⁶ represents an alkyl group having 7 to 22 carbon atoms or an alkenyl group having 7 to 22 carbon atoms.

7. A glyceryl ascorbic acid acylated derivative or its salt according to claim 6, in which R⁶ is an alkyl group having 11, 13, 15 or 17 carbon atoms.

8. A glyceryl ascorbic acid acylated derivative or its salt according to claim 1, in which R¹ represents R⁵—O—CH₂—CH(OH)—CH₂—, R² and R³ represent a hydrogen atom, and R⁴ and R⁵ represents R⁶—CO—.

9. A glyceryl ascorbic acid acylated derivative or its salt according to claim 8, in which R⁶ represents an alkyl group having 7 to 22 carbon atoms or an alkenyl group having 7 to 22 carbon atoms.

10. A glyceryl ascorbic acid acylated derivative or its salt according to claim 1, in which R¹ represents R⁵—O—CH₂—CH(OH)—CH₂—, R³ represents a hydrogen atom, and R², R⁴ and R⁵ represent R⁶—CO—.

11. A production method of a glyceryl ascorbic acid acylated derivative or its salt comprising reacting a glyceryl ascorbic acid with an acylating agent.

12. A production method of a glyceryl ascorbic acid acylated derivative or its salt according to claim 11, in which the acylating agent is an acid halide or an acid anhydride and the reaction is carried out under basic conditions.

13. A production method of a glyceryl ascorbic acid acylated derivative or its salt according to claim 11, in which the acylating agent is a carboxylic acid and the reaction is carried out under acidic conditions.

14. A cosmetic comprising the glyceryl ascorbic acid acylated derivative or its salt according to any one of claims 1 to 10.
